(19) United States
(12) Patent Application Publication
(10) Pub. No.: US 2024/0390045 A1
(43) Pub. Date: Nov. 28, 2024

(54) MEDICAL FIXING SYSTEM

(71) Applicant: BRICON GMBH, Wurmlingen (DE)

(72) Inventors: Reiter ZORAN, Emmingen-Liptingen (DE); Reitzig CLIFF-GEORG, Wurmlingen (DE); Lai YU-SHU, Taipei City (TW)

(21) Appl. No.: 18/688,582

(22) PCT Filed: Jun. 30, 2022
(86) PCT No.: PCT/EP2022/068091

§ 371 Date: Mar. 1, 2024

(30) Foreign Application Priority Data
Sep. 6, 2021 (DE) 20 2021 104 790.9

(51) Int. Cl. A61B 17/80; A61B 17/00

(52) U.S. Cl. CPC A61B 17/8057; A61B 2017/00526

(57) ABSTRACT

The invention relates to a medical fixation system (1) comprising a connecting means (3) extending in a longitudinal direction (A) and a fixing part (5) which, in the assembled state, is adapted to be brought into engagement with the connecting means (3) and to extend over a breaking point, wherein the fixing part (5) comprises a fixing body (7) with a through hole (13) which is adapted to at least partially receive the connecting means (3) in the assembled state, wherein the through hole (13) extends coaxially with a longitudinal axis (L) and is defined by a wall (15) which extends circumferentially around the longitudinal axis (L). The invention proposes that the through hole (13) further comprises at least one cavity (30a-30j) formed in the wall (15), wherein the cavity (30a-30j) is arranged to receive the connecting means (3) in the assembled state at least in sections. The invention also relates to a fixing part (5) for a fixation system (1) and a manufacturing method for manufacturing a fixing part (5).

The present invention relates to a medical fixation system comprising an axially extending connecting means, preferably a screw, and a fixation member adapted to be engaged with the connecting means in an assembled state. The fixing part has a fixing body and a through hole in the fixing body, which is arranged to at least partially receive the connecting means in the assembled state, wherein the through hole is defined by a wall and extends coaxially about a longitudinal axis. The fixing part is designed to extend over a fracture site, i.e. the bone fragments of a bone, in the assembled state and, in particular, to be fixed to a bone by at least one connecting means.

Medical fixation systems are understood to be orthopaedic fixation systems or surgical fixation systems. Such fixation systems are generally known. They are generally used for the mechanical stabilization of bone fractures. They comprise a fixing part that rests against a bone in the assembled state and extends over a fracture site, i.e. the bone fragments. It is also possible for the fixing part to extend over a fracture site at a distance from a bone. In this case, the fixing part is connected to the bone or its bone fragments by means of one or more connecting means. If several connecting means are used, the fixing part ensures the positioning of the connecting means in relation to each other. For this purpose, the fixing part has one or more through holes in its fixation body, each of which is designed to at least partially accommodate a fastener. Such a fixing part is also referred to as a bone plate. The fixing part and the connecting means are preferably coupled to each other by a force-fit and/or form-fit connection.

It is known that some types of bone fragments tend to change their position relative to the fixing part over time. This disadvantageous condition can be caused or facilitated by “slippage” of the connecting means in engagement with the fixing part. With the aim of forming a positive and non-positive connection between the respective connecting means and the fixing part and thus effectively preventing “slipping”, the connecting means and/or the fixing means often have a thread at least in sections.

The connecting means is therefore preferably a screw with at least one external thread, which can be screwed to the bone to be stabilized. Furthermore, bolts, nails or pins can also be provided as connecting means. Furthermore, the through holes of such fixing parts can have an internal thread.

Furthermore, connecting means with a self-tapping or thread-forming or self-forming external thread can be used together with a fixing part. To prevent unintentional loosening of the fastener from the through hole, the fastener preferably interacts with the through hole of the fixing part in such a way that the fixing part is plastically deformed when the fastener is inserted and, for example, a thread is cut or formed in sections so that the fastener is wedged inside the through hole of the fixing part. The external thread must engage with the wall of the respective through hole in particular in such a way that it cuts or forms an internal thread corresponding to the respective external thread in the wall of the respective through hole. This usually requires a great deal of force. Furthermore, the material selection usually has to be adapted accordingly so that the hardness of the material for the fixing part is lower than the hardness of the fastener.

There is therefore a need to make it possible to manufacture the fixing part and the fastener from the same material or a material of at least comparable hardness.

Furthermore, depending on the formation of the bone structure to be stabilized, the connecting means is inserted into the fixing part polyaxially, i.e. at an angle of inclination to suit the requirements. The angle of inclination is understood to be the angle formed between the longitudinal direction of the connecting means and the longitudinal axis of the through hole.

However, the known fixation systems with fasteners with an external thread and a fixing body with corresponding through holes only allow unidirectional insertion of the fastener, i.e. insertion in the direction of the longitudinal axis.

Various geometries of the through holes are also known from the state of the art, which are intended to enable the fastener to be inserted into the fixing part at different angles of inclination, i.e. polyaxially, for example with a thread-forming or self-cutting thread on the head section. What these solutions have in common is that the connecting means can only be brought into engagement with the fixing part by applying a great deal of force and, in particular, must be made of a harder material than the fixing part. The increased force required is not least due to the fact that it is difficult for the thread of the fastener to find a starting point on smooth walls or uninterrupted edges, for example, from which the plastic deformation of the fixing body begins. In particular, when screwing in at an angle of inclination in conical or otherwise shaped through holes, which allow the fastener to be inclined, a great deal of force is required to fix the fastener precisely in a predefined position and prevent it from sliding off the wall unintentionally.

The use of porous materials is generally unsuitable for use in the fixation of bone fractures, as their durability in particular is more difficult to determine under constant dynamic loading. The application of protrusions to prevent slippage can also only inadequately solve this problem, as this is cost-intensive to manufacture and can also lead to local hardening of the material or at least make it more difficult to screw in the fastener.

It was therefore the task of the present invention to overcome at least one of the disadvantages known from the prior art. In particular, the task of the invention was to improve the manageability of a medical fixation system and preferably to enable polyaxial insertion of the connecting means into the fixing part with reduced force, while at the same time increasing the flexibility for the surgeon in the fixation of bone fractures.

The invention solves the underlying problem by a medical fixation system according to claim 1. In particular, the invention proposes that the through hole further comprises at least one cavity provided in the wall, wherein the cavity is adapted to receive the connecting means in the mounted state at least in sections. Preferably, the fixation system has at least two connecting means. The fixing part ensures the positioning of the connecting means relative to each other. A first connecting means is in particular designed to be connected to a first bone or bone fragment and a second connecting means is in particular designed to be connected to a second bone or bone fragment.

A cavity in the sense of the invention is an empty enclosed hollow space, which extends from the wall of the through hole into the fixing body. The cavity has an opening in the area of the wall of the through hole and is spatially limited on both sides in the direction of the longitudinal axis. The cavity also extends into the fixing body in a radial direction, i.e. transversely to the longitudinal axis of the through hole. A defined volume is thus accommodated in the cavity extending from the wall into the fixing body. It should be understood that the cavity is thus also spatially limited in the circumferential direction.

At least one section of the fastener can thus be inserted through the opening of the cavity in the area of the wall and thus accommodated therein, whereby this section of the fastener is supported at least partially in the radial direction and in the direction of the longitudinal axis of the through hole downwards and upwards. This reduces the force required for plastic deformation of the fixing body, as the connecting means can be positioned more easily. The wording “downwards” or “upwards” is not to be understood as spatially restrictive and merely refers to two directions running in opposite directions and in the direction of the longitudinal axis. This makes it easier to position the fastener in the through hole.

The connecting means preferably extends in a longitudinal direction, which corresponds to the direction of the longitudinal axis when the connecting means is inserted uniaxially into the through hole and runs at an angle of inclination to the longitudinal axis when the connecting means is inserted at an angle into the through hole.

The diameter of the through hole is preferably larger than the diameter of the connecting means, in particular at least in sections, so that the connecting means can be tilted within the through hole.

The connecting means preferably has an external thread, preferably a self-forming external thread. As a self-forming or thread-forming external thread, such an external thread is designed to form a corresponding thread in the through hole by plastic deformation when the fastener is inserted into the through hole. The external thread cuts into a wall of the cavity through a rotational movement around the longitudinal axis of the fastener.

Preferably, the connecting means are designed as screws and have a shank section with a first diameter and a first external thread, which is designed to be brought into engagement with the bone. Furthermore, such screws have a head section with a second thread, which is designed to be brought into engagement with the fixing part in the assembled state.

The connecting means also preferably has a shaft section and a head section, with the diameter of the through hole being larger than the diameter of the shaft section and corresponding to the diameter of the head section. This means that the connecting means can be at least partially accommodated in the cavity, particularly in the area of the head section, and can be coupled to the fixing part in a form-fitting and force-fitting manner.

The fixing part can preferably be a plate, in particular a bone plate, or a body corresponding to the respective bone.

In particular, the cavity is designed to accommodate at least one thread in sections of a fastener with an external thread in such a way that, by accommodating the fastener in the cavity, at least a part of the fixing body surrounding the cavity extends at least partially in the direction of rotation or direction of shape of the thread or thread, so that this can plastically deform the production body with less force. This facilitates the polyaxial screwing of the fastener into the fixing part. As a result, the respective thread of the external thread quickly finds a corresponding recess in the cavity, in which the preferably self-forming thread can engage in order to come into contact with the through hole.

The connecting means and the fixing part are preferably at least partially made of a metallic material, in particular a titanium alloy.

According to a preferred embodiment, the cavity is produced by milling with a milling tool. Preferably, the milling tool is moved in the through hole at least in sections in a direction transverse to the longitudinal axis. One or more cavities with an extension transverse to the direction of the longitudinal axis can thus be created in the fixing body by milling, with the remaining part of the wall remaining in place. A corresponding thread can then be cut into this part of the wall using a fastener with an external thread, for example, without this being hindered by irregular surface structures. Milling can be easily automated and is particularly suitable for small diameters of the through hole and thicknesses of the fixing part. Milling processes belonging to the micro-machining group are particularly preferred for producing the cavity.

Preferably, the cavity has a concave shape that forms a spatially defined receiving space. The concave shape defines a receiving space and the cavity is limited by the fixing body in the axial direction, in the circumferential direction and, at least in sections, also in the radial direction. This means that in the event of an inclined position, the fastener can be accommodated at a defined position within the fixing body.

According to a preferred embodiment, the cavity is a first cavity and the through hole further comprises at least one second cavity, which is formed in the wall and is arranged at a distance from the first cavity in the circumferential direction, so that the first cavity and the second cavity are angularly offset from each other and form an offset angle between them. Thus, the connecting means can be received in a first position and a defined inclination in the first cavity and preferably be screwed, with at least one thread of the connecting means engaging at least in sections in the first cavity. Furthermore, the connecting means can be accommodated in a second position and at a defined inclination in the second cavity and preferably screwed, with at least one thread of the connecting means engaging at least in sections in the second cavity.

An offset angle of 40° to 180° is preferred, in particular 45° to 180°.

Preferably, the first cavity is arranged at a distance from the second cavity in the direction of the longitudinal axis. The distance between the first cavity and the second cavity increases the flexibility of the fixation system, particularly with regard to the possible angle of inclination of the connecting means.

According to a preferred embodiment, the first cavity and the second cavity are associated with a first plane extending transversely to the longitudinal axis, so that the first plane extends through the first cavity and the second cavity. In other words, the first cavity and the second cavity are arranged at the same height in the direction of the longitudinal axis. Thus, a defined first position and a defined second position for receiving a section of the connecting means are provided, which differ only in the direction of the inclination of the connecting means relative to the longitudinal axis.

According to a particularly preferred embodiment, the through hole also has a third cavity formed in the wall and a fourth cavity formed in the wall. The third cavity and the fourth cavity are preferably arranged at a distance from one another in the circumferential direction. Preferably, the third cavity and the fourth cavity are associated with a second plane extending perpendicular to the longitudinal axis, so that the second plane extends through the third cavity and the fourth cavity. In other words, the third cavity and the fourth cavity are arranged at the same height in the direction of the longitudinal axis. Preferably, the first plane is arranged at a distance from the second plane in the direction of the longitudinal axis. The positions of the connecting means can differ both in the direction of their inclination and in their angle of inclination relative to the longitudinal axis. This further increases the flexibility of the fixation system with regard to the angle of inclination.

Preferably, the first plane is angularly offset relative to the second plane, so that a torsion angle is defined between the first cavity and the third cavity and between the second cavity and the fourth cavity. Due to the torsion angle, the position of the connecting means differs, for example, in the first position, in which the connecting means is accommodated in the first cavity, from the third position, in which the connecting means is accommodated in the third cavity, by the direction of inclination due to the torsion angle and by the angle of inclination due to the distance between the first plane and the second plane. The same applies to the second and fourth cavities and the second and fourth positions of the connecting means that this enables. This further increases the flexibility of the fixation system with regard to the angle of inclination and, in particular, the direction of inclination.

A torsion angle of 40° to 140° is preferred, in particular 45° to 135°.

Preferably, the through hole is coaxial and symmetrical about the longitudinal axis.

According to a preferred embodiment, at least one section of the wall is designed as a helical wall. Preferably, the helical wall is designed to correspond to an external thread of the connecting means, in particular to an external thread of the head section. This facilitates the engagement of the connecting means with the through hole and enables a force-fit and form-fit connection.

Preferably, the through hole extends from a first side to an opposite second side of the fixing body and has an enlarged entry section on the first side of the fixing body. In this enlarged entry section, the connecting means can in particular also be inserted at an inclination of the longitudinal direction of the connecting means relative to the longitudinal axis of the through hole before it is received at least in sections in the one or more cavities.

Further preferably, the through hole, which extends from one or the first side to one or the opposite second side of the fixing body, has an enlarged outlet section on the second side of the fixing body. This outlet section can be provided alone or together with the inlet section. In particular, the enlarged outlet section permits an inclination of the longitudinal direction of the connecting means relative to the longitudinal axis of the through hole in the exit region of the through hole, while the connecting means is accommodated at least in sections by the cavity.

Particularly preferably, the through hole widens successively from the second side to the first side of the fixing body at an opening angle in relation to the longitudinal axis, thereby providing the enlarged inlet section. Thus, the through hole is conical at least in sections and thus has the shape of a truncated cone. For the purposes of the invention, the one or more cavities can be formed in this conically shaped section in the wall of the through hole. Thus, the connecting means can already be inserted in an inclination of the longitudinal direction of the connecting means relative to the longitudinal axis of the through hole before it is received at least in sections in the one or more cavities.

Further preferably, the through hole between the inlet section and the outlet section is conical and runs at an opening angle in relation to the longitudinal axis. The diameter of the through hole in the conically shaped section preferably reaches a maximum adjacent to the inlet section.

An opening angle of 0° to 60° is preferred, in particular 0° to 45°.

Particularly preferably, a first section and a second section of the through hole are truncated cone-shaped, whereby the enlarged inlet section is provided on the first side of the fixing body and the enlarged outlet section is provided on the second side of the fixing body. In other words, the through hole is formed as a truncated cone in the region of the inlet section and the outlet section, with the tips pointing towards each other, so that the through hole as a whole is formed similarly to an hourglass.

Preferably, the fixing part has a thickness between the first side and the second side that is equal to or greater than 0.5 mm. This provides a sufficient thickness for inserting at least one cavity.

The invention has been described above in relation to a first aspect. In a second aspect, the invention relates to a fixation member for a medical fixation system adapted to be engaged with a connecting means in an assembled state, the fixation member having a through hole adapted to at least partially receive the connecting means in the assembled state, the through hole extending coaxially with a longitudinal axis and being defined by a wall.

The invention solves the underlying problem in the second aspect in that the through hole also has at least one cavity formed in the wall, which is set up to receive the connecting means in the assembled state, at least in sections.

The fixing part according to the invention adopts the advantages of the fixation system with such a fixing part described above in relation to the first aspect. Advantages and preferred embodiments of the first aspect are also advantages and preferred embodiments of the second aspect of the invention.

In a third aspect, the invention relates to a method for producing a fixing part, in particular a method for producing a fixing part according to the second aspect of the invention, comprising the steps of: introducing a through hole into a fixing body, the through hole extending coaxially to a longitudinal axis, introducing a cavity into a wall of the through hole by milling with a milling tool, the milling tool being moved in the through hole at least in sections in a direction transverse to the longitudinal axis.

By producing a fixing part with a cavity in the wall of a through hole, the method according to the invention adopts the advantages described above in relation to the first and second aspects of the invention. Preferred embodiments of the first and second aspects of the invention are also preferred embodiments of the third aspect of the invention and vice versa.

Further advantages, features and aspects of the invention are shown in the following description and in the drawings.

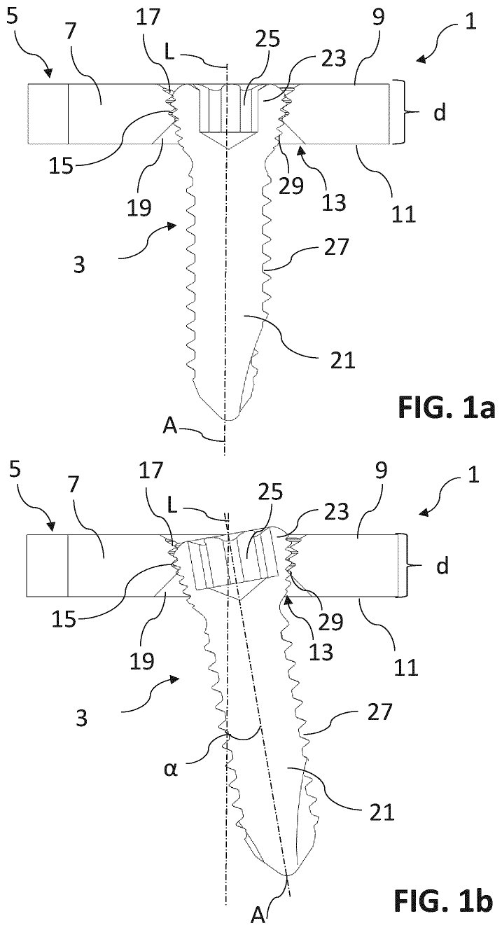

FIG. 1a shows a medical fixation system according to a first embodiment in a first position in a sectional view;

FIG. 1b shows the fixation system according to FIG. 1a in a second position in a sectional view;

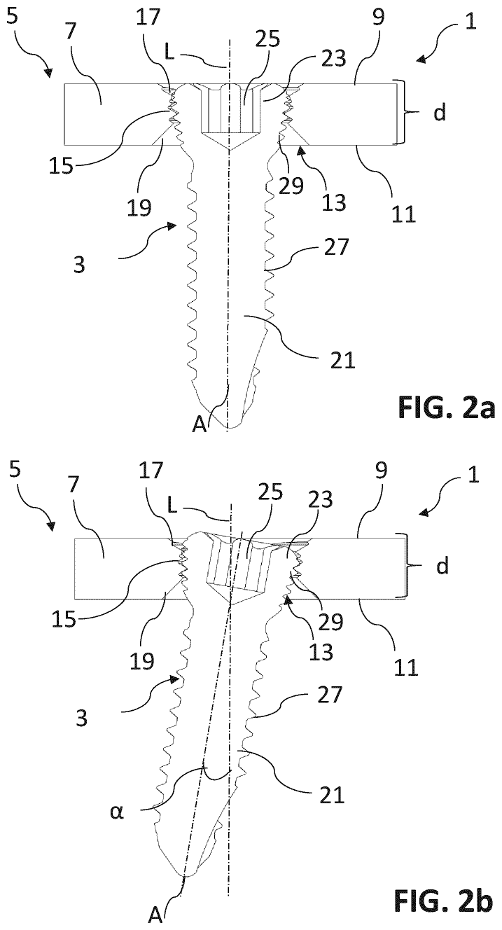

FIG. 2a shows the fixation system according to a second embodiment in a first position in a sectional view;

FIG. 2b shows the fixation system according to FIG. 2a in a second position a sectional view;

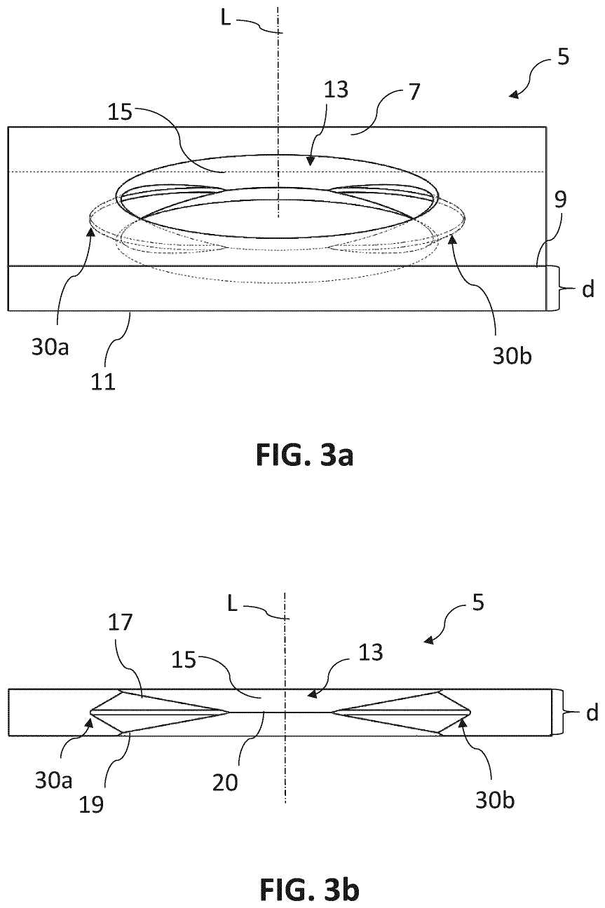

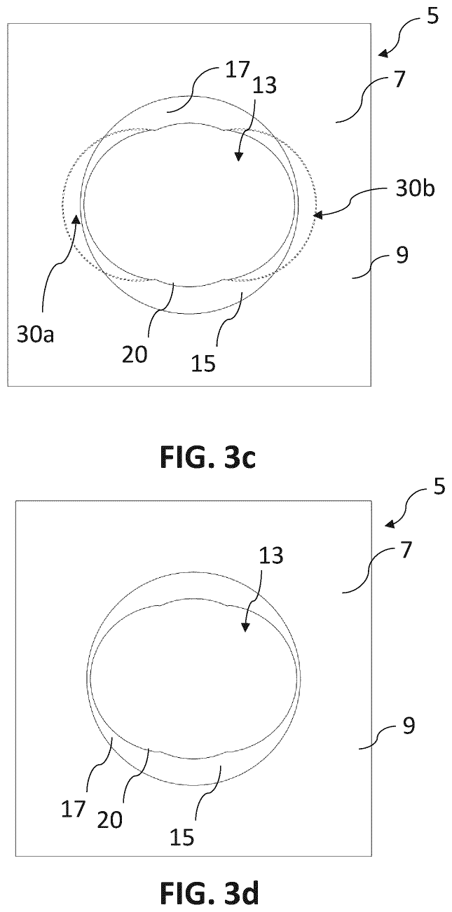

FIG. 3a shows a fixing part according to a first embodiment in a perspective view;

FIG. 3b shows the fixing part according to FIG. 3a in a first sectional view;

FIG. 3c shows the second sectional view of the fixing part shown in FIG. 3a;

FIG. 3d shows a plan view of the fixing part according to FIG. 3a;

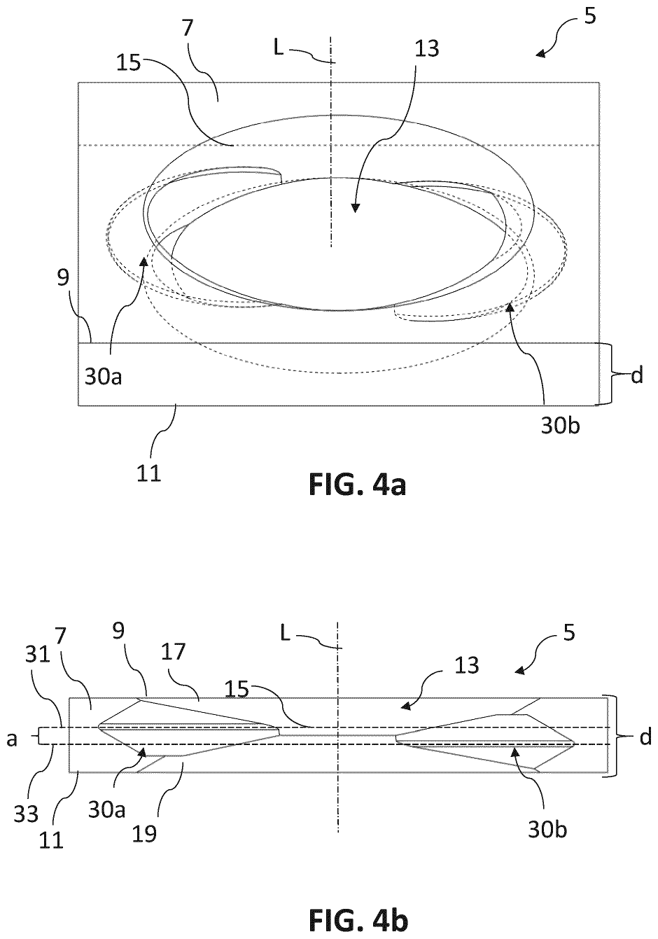

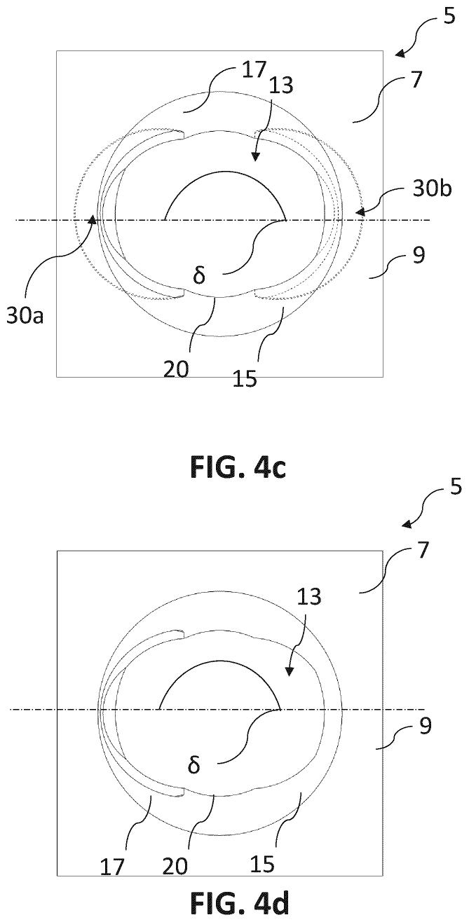

FIG. 4a shows a fixing part according to a second embodiment in a perspective view;

FIG. 4b shows the fixing part according to FIG. 4a in a first sectional view;

FIG. 4c shows the fixing part according to FIG. 4a in a second sectional view;

FIG. 4d shows the fixing part according to FIG. 4a in a plan view;

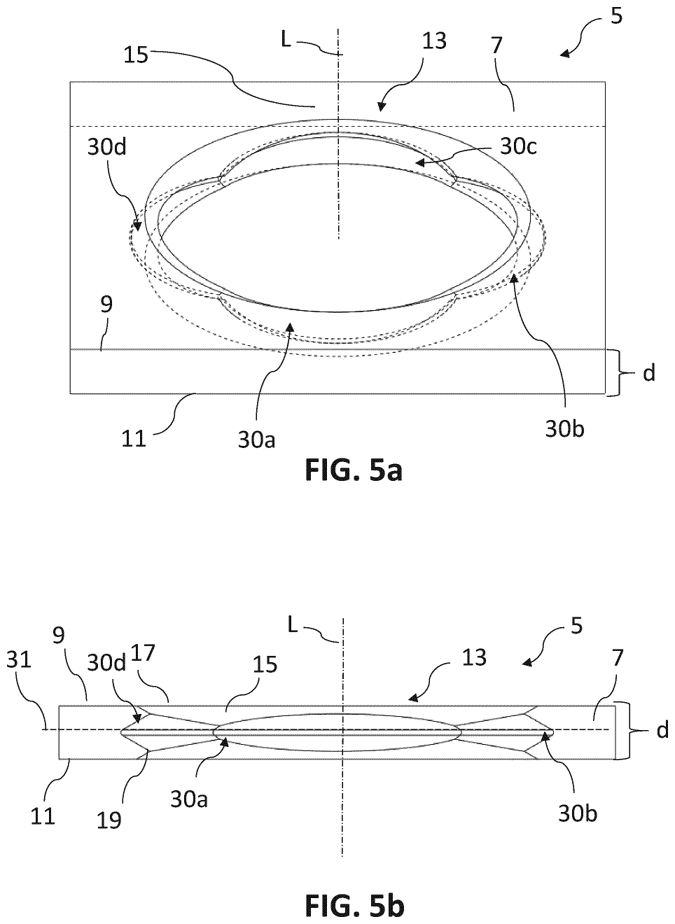

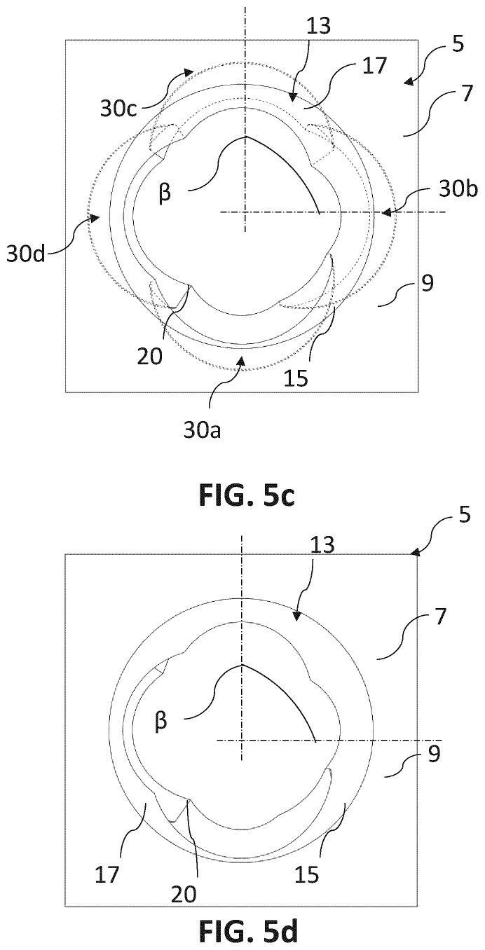

FIG. 5a shows a fixing part according to a third embodiment in a perspective view;

FIG. 5b shows the fixing part according to FIG. 5a in a first sectional view;

FIG. 5c shows the fixing part according to FIG. 5a in a second sectional view;

FIG. 5d shows the fixing part according to FIG. 5a in a plan view;

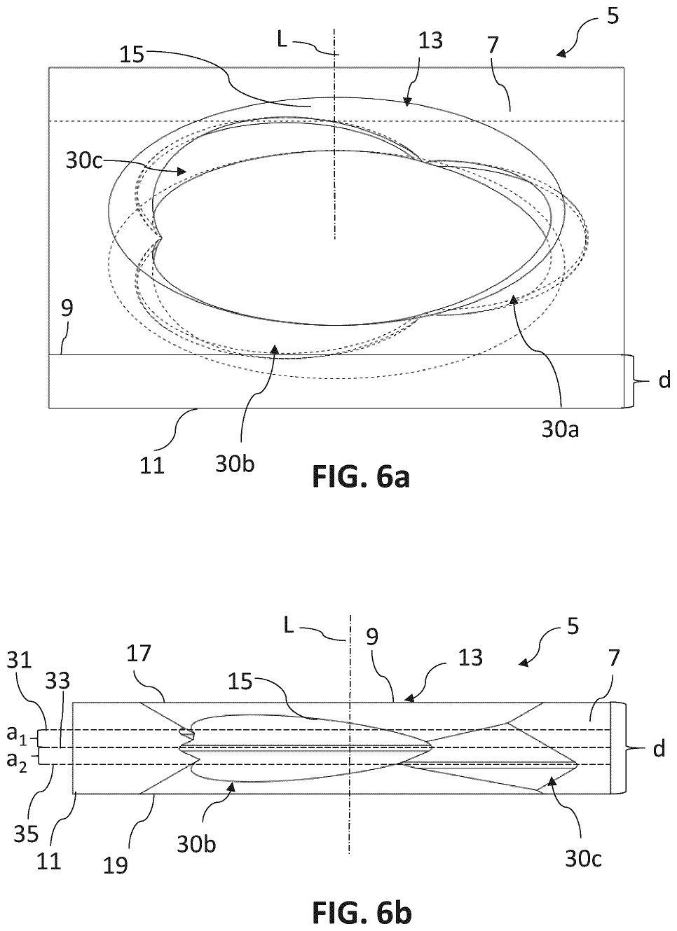

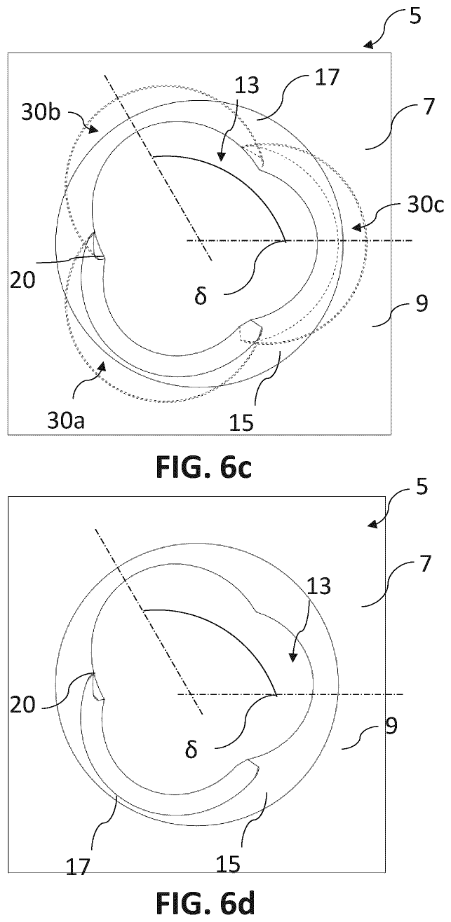

FIG. 6a shows a fixing part according to a fourth embodiment in a perspective view;

FIG. 6b shows the fixing part according to FIG. 6a in a first sectional view;

FIG. 6c shows the fixing part according to FIG. 6a in a second sectional view;

FIG. 6d shows the fixing part shown in FIG. 6a a plan view;

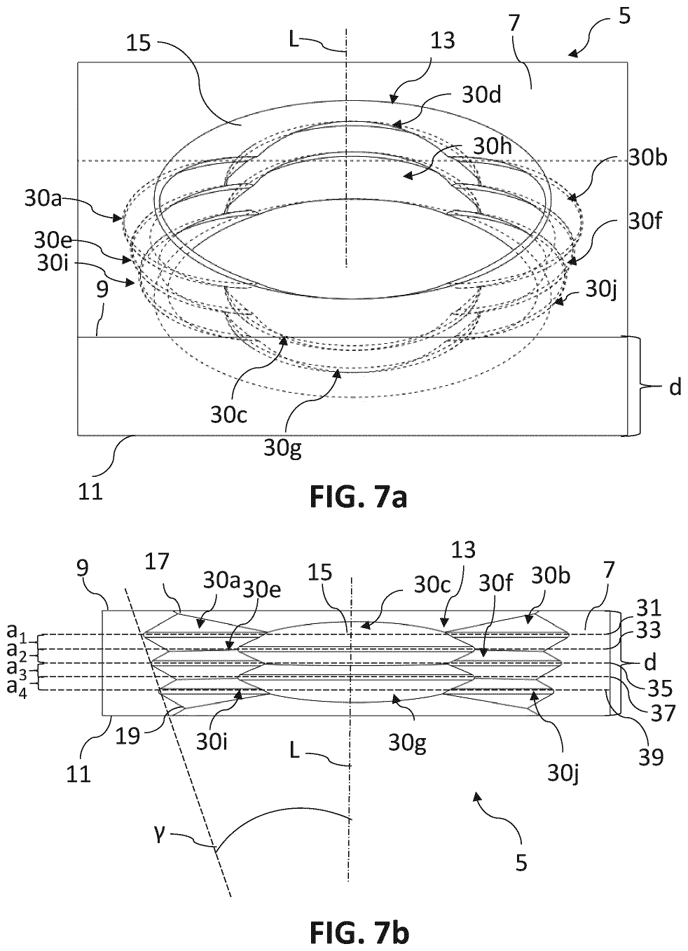

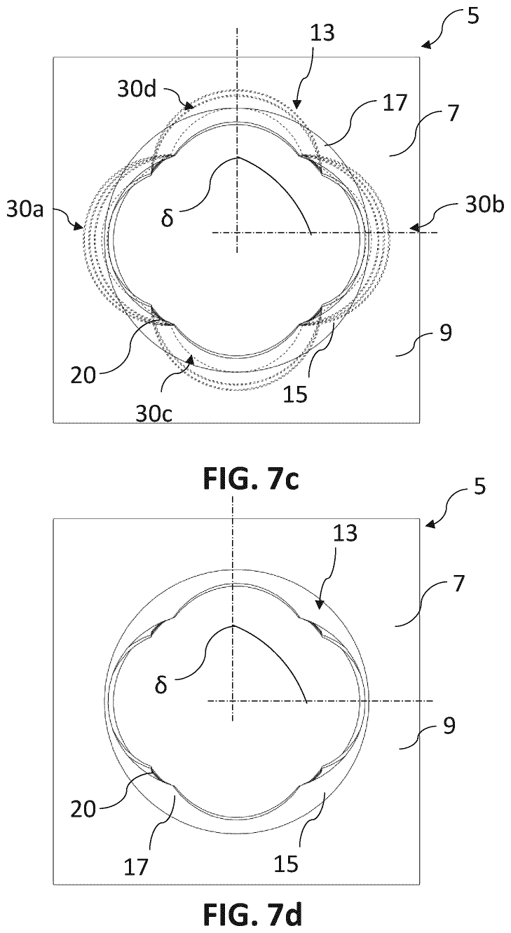

FIG. 7a shows a fixing part according to a fifth embodiment in a perspective view;

FIG. 7b shows the fixing part according to FIG. 7a in a first sectional view;

FIG. 7c shows the fixing part shown in FIG. 7a in a second sectional view;

FIG. 7d shows the fixing part as shown in FIG. 7a in a plan view;

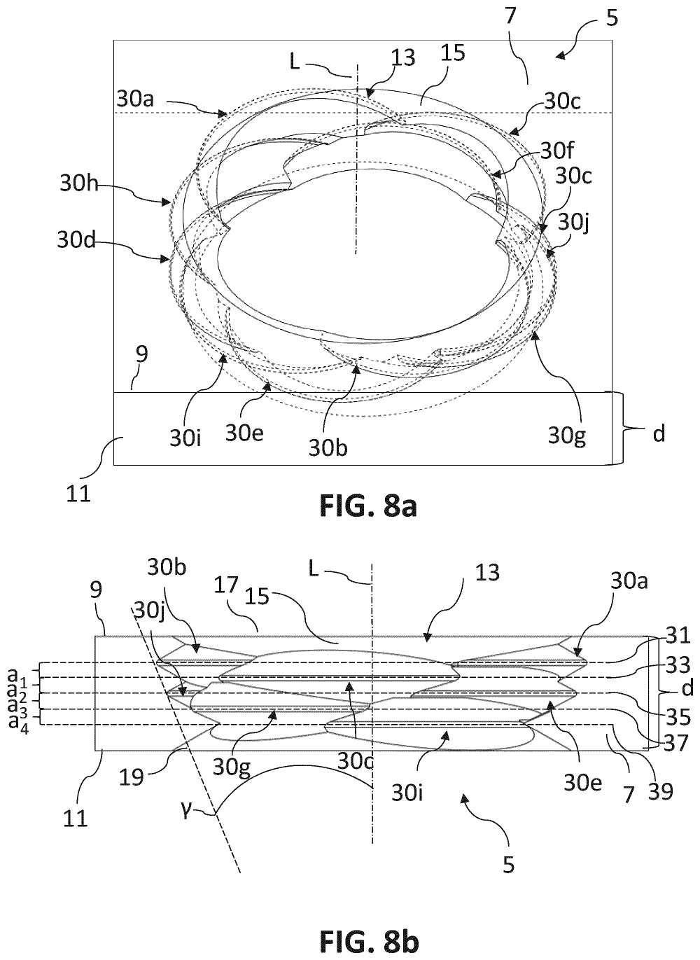

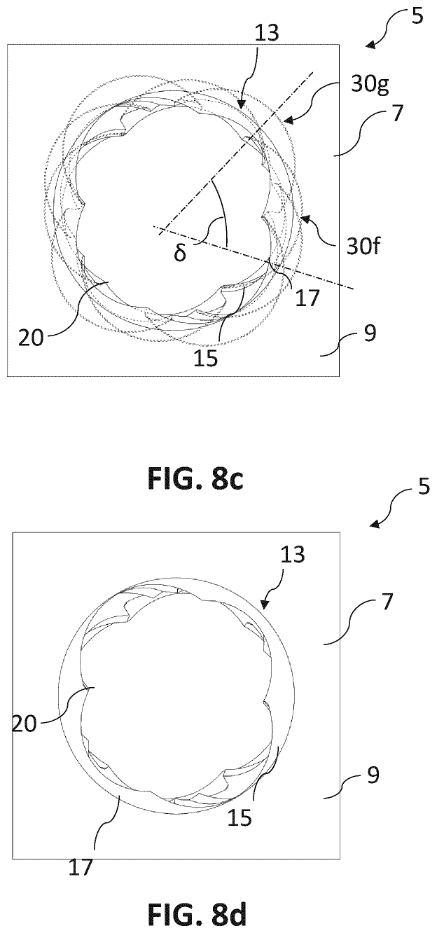

FIG. 8a shows a fixing part according to a sixth embodiment in a perspective view;

FIG. 8b shows the fixing part according to FIG. 8a in a first sectional view;

FIG. 8c shows the fixing part according to FIG. 8a in a second sectional view;

FIG. 8d shows the fixing part shown in FIG. 8a in a plan view.

FIGS. 1a and 1b show a first embodiment of the medical fixation system 1 according to the invention.

The fixation system 1 has a connecting means 3 and a fixing part 5. FIGS. 1a and 1b show the fixation system 1 in an assembled state, in which the connecting means 3 is engaged with the fixing part 5.

In FIG. 1a, the connecting means 3 is shown in a first position, in which it extends along a longitudinal direction A corresponding to the longitudinal axis L. In FIG. 1b, the connecting means 3 is shown in a second position, in which the longitudinal direction A of the connecting means 3 is inclined at an angle α to the longitudinal axis L.

The fixing part 5 has a fixing body 7 with a thickness d in the direction of the longitudinal axis L.

The fixing body 7 has a first side 9 and a second side 11 spaced apart in the direction of the longitudinal axis L. A through hole 13, which is formed in the fixing body 7, extends from the first side 9 to the second side 11. The through hole 13 thus extends in the direction of the longitudinal axis L.

The through hole 13 is designed to correspond to the connecting means 3 and has a wall 15, which engages with the connecting means 3 in the assembled state. The through hole 13 also has an inlet section 17 on the side of the first side 9 and an outlet section 19 on the side of the second side 11.

The through hole 13 has a central section 20 arranged between the inlet section 17 and the outlet section 19. Preferably, the through hole 13 is enlarged in cross-section in the region of the inlet section 17 and/or in the region of the outlet section 19 in a plane extending perpendicular to the longitudinal axis L, so that the middle section 20 represents the region with the smallest cross-sectional area.

In order to bring the fastener 3 into engagement with the through hole 13, the fastener is inserted into the inlet section 17, passed through the middle section 20 and finally exits the outlet section 19, at least in sections. In the assembled state, the fastener 3 is received at least in sections in the through hole 13.

In a preferred embodiment, the connecting means 3 is shown as a screw extending in a longitudinal direction A. The connecting means 3 comprises a shank portion 21 and a head portion 23. The head portion 23 preferably has a recess 25, which is designed to cooperate with a tool for engaging the connecting means 3 with the fixing part 5.

The fastener 3 preferably has a first external thread 27 in the region of the shank section 21 and further preferably a second external thread 29 in the region of the head section 23. The second thread 29 can preferably be a self-forming thread, which forms a thread in the through hole 13 by plastic deformation.

The connecting means 3 has a larger diameter in the area of the head section 23 than in the area of the shaft section 21. The through hole 13 has a diameter or a minimum diameter that is adapted to the diameter of the head section 23. This ensures that the shank section 21 can be moved through the through hole 13 in order to bring the fastener 3 into engagement with the fixing part 5 in an assembled state. The head section 23 preferably has a longitudinal extension that corresponds to the thickness d of the fixing part 5.

The through hole 13 preferably has at least one cavity (see FIGS. 7a to 7d), which is designed to accommodate the fastener 3, in particular the head section 23, at least in sections in the assembled state. Through the cavity (not shown), the connecting means 3 can also be brought into engagement with the through hole 13 of the fixing part 5 at an angle of inclination α, as shown in FIG. 1b.

The embodiment example shown in FIGS. 2a and 2b differs from the embodiment example described above only in the design of the through hole 13 and in particular the cavities (see FIGS. 8a to 8d).

As shown in particular in FIGS. 1b and 2b, the connecting means 3 can be accommodated in the through hole 13 at various angles of inclination α. In particular, the external thread 29 of the head section 23 is positively and non-positively engaged with the wall 15 of the through hole 13.

FIGS. 3a to 3d show a first preferred embodiment of the fixing part 5 according to the invention.

The fixing part 5 has a fixing body 7 with a first side 9 and a second side 11, wherein a through hole 13 extends between the first side 9 and the second side 11 in the direction of a longitudinal axis L.

The through hole 13 is defined by a wall 15, which extends around the longitudinal axis L in a circumferential direction. The through hole 13 is therefore coaxial with the longitudinal axis L.

The through hole 13 has a central section 20 arranged between the inlet section 17 and the outlet section 19.

In the embodiment example shown, the wall 15 is frustoconical or conical at least in sections, so that the inlet area 17 provides an enlarged cross-sectional area compared to the central section 20 in a plane orthogonal to the longitudinal axis L.

In the central area 20, the through hole 13 thus has a reduced diameter in sections. The through hole 13 further comprises a first cavity 30a and preferably a second cavity 30b, which are formed between the first side 9 and the second side 11 in the region of the wall 15. Because the first cavity 30a and the second cavity 30b are arranged in the central region 20, an enlarged receiving space for the connecting means is provided there locally.

In the embodiment example shown, the first cavity 30a is arranged in a plane extending orthogonally to the longitudinal axis L at an offset angle β of 180° to the second cavity 30b, as shown in particular in FIGS. 3c and 3d.

In FIG. 3c, the fixing part 5 is shown in a first sectional view through a plane extending orthogonally to the longitudinal axis L, which extends centrally in the direction of the longitudinal axis L, i.e. through the middle section 20 and the fixing body 7. The inlet section 17 has an enlarged cross-section compared to the central section 20, with the central section 20 having two additional receiving spaces for receiving at least one section of the connecting means 3 (see FIGS. 1a to 2b) due to the cavities 30a, 30b provided.

As shown in particular in FIG. 3b, the cavities 30a, 30b are arranged at the same height in the direction of the longitudinal axis L, i.e. at the same level.

The through hole 13 is formed coaxially to the longitudinal axis L and the first cavity 30a and the second cavity 30b are arranged opposite each other in the through hole 13.

The embodiment example shown in FIGS. 4a to 4d differs from the first embodiment example of the fixing part 5 according to the invention shown in FIGS. 3a to 3d in the arrangement of the first cavity 30a and the second cavity 30b. The first cavity 30a is arranged at a distance a from the second cavity 30b in the direction of the longitudinal axis L in the embodiment example shown in FIGS. 4a to 4d.

The first cavity 30a is arranged on a first plane 31, which extends orthogonally to the longitudinal axis L, and the second cavity 30b is arranged on a second plane 33, which also extends orthogonally to the longitudinal axis L. The first plane 31 is arranged at a distance a in the direction of the longitudinal axis L at a distance from the second plane 33. The first plane 31 and the second plane 33 are offset from each other at a torsion angle δ of 180°, so that the first cavity 30a is arranged opposite the second cavity 30b.

Reference is also made to the detailed description of the design of the fixing part 5 with reference to FIGS. 3a to 3d.

FIGS. 5a to 5d show a third preferred embodiment example of the fixing part 5. The embodiment example differs from the embodiment example shown above in FIGS. 3a to 3d in the number of cavities. In the embodiment example shown, the through hole 13 has a first cavity 30a, a second cavity 30b, a third cavity 30c and a fourth cavity 30d, which are arranged on a common plane and thus at the same height in the direction of the longitudinal axis L according to the view shown in FIG. 5b. In particular, the common plane 31 is arranged centrally in relation to the thickness d of the fixing body 7, i.e. in the region of the central section 20.

As shown in particular in FIGS. 5c and 5d, the first cavity 30a and the second cavity 30b are angularly offset and include an offset angle β of 90° between them. Furthermore, an offset angle β of 90° is also formed between the second cavity 30b and the third cavity 30c, which is shown here as an example for all of the offset angles β. Furthermore, an offset angle β of 90° is also formed between the third cavity 30c and the fourth cavity 30d as well as between the fourth cavity 30d and the first cavity 30a.

Reference is also made to the detailed description of the design of the fixing part 5 with reference to FIGS. 3a to 3d.

In the embodiment example shown in FIGS. 6a to 6d, the through hole 13 has cavities which are assigned to different planes, as also shown in particular in FIGS. 4a to 4d. The embodiment example shown in FIGS. 6a to 6d differs from the embodiment example shown in FIGS. 4a to 4d in the number of cavities.

The through hole 13 has a first cavity 30a, a second cavity 30b and a third cavity 30c in the wall 15. The first cavity 30a is assigned to a first plane 31, the second cavity 30b is assigned to a second plane 33 and the third cavity 30c is assigned to a third plane 35. The first plane 31 and the second plane 33 are arranged at a distance a₁ from each other in the direction of the longitudinal axis L. The second plane 33 and the third plane 35 are arranged at a distance a₂ from each other in the direction of the longitudinal axis L.

As shown in particular in FIGS. 6c and 6d, the first plane 31 is arranged at a torsion angle δ to the second plane 33 and the second plane 33 is arranged at a torsion angle δ to the third plane 35. In FIGS. 6c and 6d, the angle of rotation δ between the second plane 33 and the third plane 35 is shown as an example.

Reference is also made to the detailed description of the design of the fixing part 5 with reference to FIGS. 3a to 3d.

The fifth embodiment example of the fixing part 5 according to the invention shown in FIGS. 7a to 7d differs from the embodiment examples described above in particular in the design of the through hole 13.

A total of ten cavities 30a to 30j are formed in the wall 15 of the through hole 13. A first cavity 30a and a second cavity 30b are assigned to a first level 31. A third cavity 30c and a fourth cavity 30d are assigned to a second plane 33. The second plane 33 is arranged at a distance a₁ in the direction of the longitudinal axis L at a distance from the first plane 31. A fifth cavity 30e and a sixth cavity 30f are assigned to a third plane 35. The third plane 35 has a distance a₂ from the second plane 33 in the direction of the longitudinal axis L. A seventh cavity 30g and an eighth cavity 30h are assigned to a fourth plane 37. The fourth plane 37 has a distance as in the direction of the longitudinal axis L from the third plane 35. A ninth cavity 30i and a tenth cavity 30j are assigned to a fifth plane 39. The fifth plane 39 has a distance a₄ from the fourth plane 37 in the direction of the longitudinal axis L.

As shown in particular in FIG. 7b, the through hole 13 widens successively in the direction of the first side 9 in the direction of the longitudinal axis L at an opening angle γ. Thus, the cavities 30a-30j, which are each located on different planes 31 to 39 and have an offset angle β=0, are also arranged offset to one another at the opening angle γ in the sectional view shown in FIG. 7b.

The through hole 13 with a total of ten cavities 30a to 30j thus widens in the direction of the first side 9 at an opening angle γ. This provides a maximum cross-section in the inlet section 17 in the area of the first plane 31. The cross-section continues to reduce from the first plane 31 towards the fifth plane 39. Thus, adjacent to the inlet section 17, a maximized receiving space is provided for the connecting means 3 (see FIGS. 1a to 1b), into which the respective connecting means 3 can also move at an angle of inclination α, at least in sections, in order to engage with the fixing part 5 in an assembled state.

Reference is also made to the detailed description of the design of the fixing part 5 with reference to FIGS. 3a to 3d.

As shown in particular in FIGS. 7a and 7b, the two cavities 30a to 30j, each assigned jointly to one of the planes 31 to 37, are each arranged at an offset angle β of 180° to one another, i.e. opposite one another in the view shown in FIGS. 7c and 7d.

As can also be seen from FIGS. 7c and 7d, the second plane 33 is arranged at a torsion angle δ of 90° relative to the first plane 31, the third plane 35 is arranged at a torsion angle δ of 90° relative to the second plane 33, the fourth plane 37 is arranged at a torsion angle δ of 90° relative to the third plane 35 and the fifth plane 39 is arranged at a torsion angle δ of 90° relative to the fourth plane 37. In FIGS. 7c and 7d, only the angle of rotation γ between the first plane 31 and the second plane 33 is shown as an example to provide a better overview.

This means that the respective connecting means 3 (see FIGS. 1a to 2b) can be tilted in different directions and can also be brought into engagement with the fixing part 5 at different angles of inclination α due to the cavities at different levels. In this way, the flexibility for fixing a bone fracture is further increased by the fixing part 5.

The embodiment example shown in FIGS. 8a to 8d differs from the embodiment example shown above in FIGS. 7a to 7d in the angle of rotation γ of the planes 31 to 39 relative to one another. Whereas in FIGS. 7a to 7d a rotation angle δ of 90° between two adjacent planes was provided in each case, in the embodiment example shown in FIGS. 8a to 8d a rotation angle γ of 75° between two adjacent planes is provided in each case, as shown in particular in FIG. 8c.

In the view shown in FIG. 8c, the angle of rotation δ between the first plane 31 and the second plane 33 is shown as an example. The torsion angle δ is 75° in the embodiment example shown, whereas the torsion angle in the embodiment example shown in FIGS. 7a to 7d was 90°. This enables a finer alignment of the connecting means 3 (see FIGS. 1a to 2b) in relation to the fixing part 5.

Reference is also made to the detailed description of the design of the fixing part 5 with reference to FIGS. 3a to 3d.

### REFERENCE SIGN

1 Medical fixation system
3 Fastener, screw
   5 Fixing part
   7 Fixing body
   9 first page
   11 Second page
13 Through hole
15 Wall
17 Entry section
19 Exit section
20 middle section
   21 Shaft section
   23 Header section
   25 Recess
27 first external thread
29 Second external thread
30a-30j Cavity
31 first level
33 Second level
35 Third level
37 fourth level
39 fifth level
α Angle of inclination
β Offset angle
γ Opening angle
δ Twist angle
   L Longitudinal axis
   A Longitudinal direction
d Thickness
a, a₁, a₂, a₃, a₄ Distance

1. Medical fixation system, comprising:
a connecting means extending in a longitudinal direction, and
a fixing part which is arranged to be brought into engagement with the connecting means in an assembled state and to extend over a breaking point,
wherein the fixing part has a fixing body and a through hole in the fixing body which is arranged to at least partially receive the connecting means in the assembled state,
wherein the through hole extends coaxially to a longitudinal axis and is defined by a wall which extends in a circumferential direction around the longitudinal axis, and
wherein the through hole has at least one cavity formed in the wall, wherein the at least one cavity is designed to accommodate the connecting means in the assembled state, at least in sections.

2. The medical fixation system according to claim 1, wherein the at least one cavity is produced by milling with a milling tool, the milling tool being moved in the through hole at least in sections in a direction transverse to the longitudinal axis.

3. The medical fixation system according to claim 1, wherein the at least one cavity has a concave shape which forms a spatially defined receiving space,
wherein the receiving space is limited in a circumferential direction.

4. The medical fixation system according to claim 1 wherein
the at least one cavity comprises a first cavity and the through hole comprises at least one second cavity formed in the wall and arranged at a distance from the first cavity in a circumferential direction, so that the first cavity and the second cavity are angularly offset from each other and form an offset angle between them,
wherein the second cavity is arranged in a direction of the longitudinal axis at a distance from the first cavity, and
wherein the offset angle is 40° to 140°.

5. The medical fixation system according to claim 4, wherein
the first cavity and the second cavity are assigned to a first plane which extends perpendicular to a longitudinal axis so that the first plane runs through the first cavity and the second cavity,
wherein through hole comprises a third cavity formed in the wall and a fourth cavity formed in the wall and arranged at a distance from the third cavity in the circumferential direction,
wherein the third cavity and the fourth cavity are associated with a second plane which extends perpendicular to the longitudinal axis so that the second plane passes through the third cavity and the fourth cavity, and
wherein the first plane is spaced apart from the second plane in the direction of the longitudinal axis.

6. The medical fixation system according to claim 5 wherein
the first plane is angularly offset relative to the second plane so that a torsion angle is defined between the first cavity and the third cavity and between the second cavity and the fourth cavity,
wherein the torsion angle is 40° to 140°.

7. The medical fixation system according to claim 1 wherein
at least one section of the wall is designed as a helical wall, and/or
the connecting means has an external thread.

8. The medical fixation system according to claim wherein
the through hole extends from a first side to an opposite second side of the fixing body and has an enlarged inlet section on the first side of the fixing body, and/or
the through hole extends from a first side to an opposite second side of the fixing body and has an enlarged outlet section on the second side of the fixing body.

9. The medical fixation system according to claim 8, wherein
the through hole widens successively from the second side to the first side of the fixing body at an opening angle with respect to the longitudinal axis thereby providing the enlarged inlet section,
wherein the opening angle is 0° to 60°.

10. The medical fixation system according to claim 1 wherein a diameter of the through hole is larger, at least in sections, than a diameter of the connecting means so that the connecting means can incline within the through hole.

11. The medical fixation system according to claim 1 wherein
the connecting means is formed as a screw and comprises a shank portion having a first diameter and a first external thread adapted to be engaged with a bone, and a head portion having a second thread adapted to be engaged with the fixing member in the assembled state.

12. The medical fixation system according to claim 1 wherein
the connecting means and the fixing part are formed at least partially from a metallic material.

13. A fixation member for a medical fixation system which is adapted to be engaged with a connecting means in an assembled state,
wherein the fixation member has a through hole which is arranged to at least partially receive the connecting means in the assembled state,
wherein the through hole extends coaxially to a longitudinal axis and is defined by a wall,
wherein the through hole has at least one cavity in the wall, wherein the at least one cavity is designed to receive the connecting means at least in sections in the assembled state.

14. A fixation member according to claim 14, which is produced by a method comprising the steps of:
insertion of a through hole in a fixing body wherein the through hole extends coaxially to a longitudinal axis,
making a cavity in a wall of the through hole by milling with a milling tool, the milling tool being moved in the through hole at least in sections in a direction transverse to the longitudinal axis.

15. Manufacturing method for a fixation member according to claim 13, comprising the steps of:
insertion of a through hole in a fixing body wherein the through hole extends coaxially to a longitudinal axis,
making a cavity in a wall of the through hole by milling with a milling tool, the milling tool being moved in the through hole at least in sections in a direction transverse to the longitudinal axis.

16. The medical fixation device of claim 8 wherein at least a first section and a second section of the through hole are frustoconical, wherein the enlarged inlet section is provided on the first side of the fixing body and the enlarged outlet section is provided on the second side of the fixing part.

17. The medical fixation device of claim 1 wherein the connecting means is a screw.

18. The medical fixation device of claim 11 wherein the fixing member has a thickness between the first side and the second side which is equal to or greater than 0.5 mm.

19. The medical fixation device of claim 2 wherein the through hole is symmetrical about the longitudinal axis.
